(19) United States
(12) Patent Application Publication
(10) Pub. No.: US 2009/0068292 A1
(43) Pub. Date: Mar. 12, 2009

(54) Therapeutic herb cosmetic composition

(76) Inventors: Liixan Jiang, Tampa, FL (US); Calvin Cao, Tampa, FL (US)

Correspondence Address: Lixian Jiang, Suite 901, 2203 North Lois Ave., Tampa, FL, 33607, US

(21) Appl. No.: 11/900,604

(22) Filed: Sep. 12, 2007

(51) Int. Cl. A61K 36/00

(52) U.S. Cl. 424/725

(57) ABSTRACT

The current invention discloses an herb composition to treat burn, sun burn, physical injury, dermatitis, or to promote skin stem cell proliferation, increase collagen synthesis, increase epidermal thickness, and increase desquamation of normal skin resulting in smoother, younger looking skin. The composition comprises of herb extract from Polygonum cuspidatum; Phellodendron amourense Ruprecht; Forsythia suspens; Dryobalanops camphora and alpha-hydroxy acid.

### FIELD OF THE INVENTION

The present invention discloses a herb composition to stimulate skin stem cell to grow, which could be used to treat burn injury, or wrinkle as a cosmetic topical application.

### BACKGROUND OF THE INVENTION

The dried root of Polygonum cuspidatum SIEB. et ZUCC. (Polygonaceae) is a well-known traditional drug in China as well as in Japan. It has been widely used for the treatment of suppurative dermatitis, gonorrhea, favus athlete's foot, and hyperlipidemia in Chinese folk medicine (Kai XIAO, Chem. Pharm. Bull. 50(5) 605-08 (2002))

It was demonstrated that extract from Polygonum cuspidatum Root has been found to prevent tumor growth (Yoshiyuki Kimura, Journal of Nutrition. 2001; 131:1844-1849), and it is also revealed that extract of Polygonum cuspidatum Root has antioxidant effect (Hsu C Y, Biol Res. 2007; 40(1):13-21.) This anti-oxidant effect may be activated by inhibition of inducible nitric oxide synthase and cyclooxygenase-2 gene expressions (Kim K W, et al. Vascul Pharmacol. 2007 August-September; 47(2-3):99-107), or by inhibition of bacteria viability (Song J H, Arch Oral Biol. 2006 December; 51(12):1131-40).

It was found extract from Polygonum cuspidatum can enhance cell proliferation (Matsuda H, Bioorg Med Chem Lett. 2001 Jul. 23; 11(14):1839-42). Interesting, it also shows that this herb can inhibit angiogenesis (Wang S, Life Sci. 2004 Apr. 2; 74(20):2467-78). And on the other side, Polygonum cuspidatum has the potential to stimulate differentiation of keratinocytes in the epidermal layer.

Phellodendron amourense Ruprecht is another China traditional herb listed as used 100 top herbs in traditional herb medicine. Recently, scientists found the extract from Phellodendron amourense Ruprecht has the capability to whiten skin (US 2007/0009455). The bark of Phellodendron amurense, is reportedly used as an antibacterial, antirheumatic, aphrodisiac, bitter stomachic, cholagogue, diuretic, expectorant, febrifuge, hypoglycemia, ophthalmic, skin vasodilator, tonic, and central nervous stimulant (CNS). (Him-Che Yeung, Handbook of Chinese Herbs and Formulas, Institute of Chinese Medicine, Los Angeles 1985; Rev. G. A. Stuart, Chinese Materia Medica, Tapei southern Materials Centre; U.S. Pat. No. 5,344,648). When it is taken internally, it can be used to treat acute diarrhea, dysentery, jaundice, including trichomonas, acute urinary tract infections, enteritis, bolis, abscesses, night sweats, and skin diseases (The Encyclopedia of Medicinal Plants; Encyclopedia of Herbs and their Uses).

Dryobalanops camphora is a tree from in Asian countries, and it is also known as Borneo camphor, Malay camphor, and camphol (US 2004/0202731). It was reported that the extract of Dryobalanops camphora can inhibit catecholamine secretion mediated by nicotinic receptor (Oh K S, Pharmacol Res 2000; 42(6):559-64). This pathway is regulated by non-competitive inhibition of calcium influx into cells (T J Park, Biochem Pharmacol, 2003, 83-90), which can prevent cell from death caused by physical injury, or chemical toxin as a common knowledge for a people in the art. When it is used as an ingredient in topical medication, Borneo Camphor can treat dermatitis (US 2005/0260289). The same is true when it is used in soft drink. In addition, the extract of Dryobalanops camphora is a strong antioxidant agent, and unstable in normal environment since it is easy for it oxidizes with oxygen (US 2004/0202731). Therefore, it may be useful to use the extract of Dryobalanops camphora in anti-aging industry.

Forsythia suspense is another traditional herb medicine, and it has been demonstrated to have strong anti-bacterial, antivirus and anti-inflammatory effect. When it is applied on the top of skin, Forsythia suspense can treat dermatological disorders such as psoriasis (J M Prieto, Journal of Pharmacy and Pharmacology, 2003, 55(9), 1275-1282). Extract from Forsythia suspense can promote wound healing in animal model, and this extract can inhibit abnormal extra-cellular matrices accumulation (U.S. Pat. No. 6,068,845), which could prevent scar formation. The component, Polycyclic Triterpene Carboxylic Acid (PTCA), from Forsythia suspense can be used to treat or prevent normal, but undesirable, skin conditions selected from the group consisting of wrinkling, sagging, photodamage skin, dry skin and age spots and soothing sensitive skin (U.S. Pat. No. 6,440,434).

However, there are no former publications or prior arts disclosed a formula which adopts the advantages of the said herb extract, and treat skin disorders as well as works as cosmetics. The present invention discloses a cosmetic herb composition, which could improve the facial appearance. In addition, the current composition could be used to treat burn, physical injury and sun burn as a topical medicine.

### SUMMARY

The present discloses a cosmetic herb composition for facial appearance purpose to treat or prevent wrinkling, sagging, photo-damage skin, dry skin and age spots and soothing sensitive skin. This herb composition can also be used to treat dermatitis or burn or other physical injury of skin.

On one embodiment, the composition includes Polygonum cuspidatum, an antioxidant agent, cell proliferation and keratinocytes differentiation promoters. When it was used with alpha-hydroxy acid, Polygonum cuspidatum can enhance can enhance cell proliferation and increase ceramide biosynthesis in keratinocytes, increase epidermal thickness, and increase desquamation of normal skin resulting in smoother, younger looking skin. Therefore, it is preferred to include alpha-hydroxy acid in the composition.

On another embodiment, the herb composition further has herbs comprising of extract from Phellodendron amourense Ruprecht, Dryobalanops camphora, and Forsythia suspense. These herb components are either anti-bacteria agents or anti-oxidant or modulator of extra-cellar matrix, which is beneficial to improve facial appearance. In addition, when these topical medications apply on skin wound, it helps wound healing and also prevent skin from infection caused by bacteria and viruses.

### DETAILED DESCRIPTION

China herb medicine has developed more than 5000 years, and until today it still works and some herbs have been extracted and applied in clinic in western countries. The present invention discloses an herb composition for cosmetics use, and it also has therapeutic function to treat burn, sun burn, physical injury and dermatitis.

One embodiment of the invention includes Polygonum cuspidatum, which is a perennial species with spreading rhizomes and numerous reddish-brown, freely branched stems. The dried root and stem of Polygonum cuspidatum, also called Hu chang, are used in traditional Chinese medicine to treat high cholesterol and other conditions (Huang KC. The pharmacology of Chinese herbs, 2^{nd} ed. CRC Press, Boca Raton, 1999: 126). This invention found that the extract of this plant has the effect to stimulate collagen synthesis, and promote wound healing and can be used to eliminate wrinkles. Preferably, the extract of the plant has an herb supplement of alpha-hydroxy acid.

On another embodiment, the invention discloses a herb composition comprising of Phellodendron amourense Ruprecht, which is an anti-inflammatory medicine to treat diarrhea, typically skin sensitivity or other condition. When it is used external, it can treat bruises and sprains. It also has anti-fungus function when it applies on the top of skin. Our recent study reveals that when it is used together with Polygonum cuspidatum, Phellodendron amourense Ruprecht has strong potential to stimulate stem cell proliferation at the dermal layers, and it also promote skin collagen synthesis and secretion.

Another embodiment of this invention includes Dryobalanops camphora, a large tree with lax, smooth branches. The bark of this tree contains some chemical can relieve pain and cause sedative. Our research also demonstrated that extract from this plant also has the function to promote cell proliferation and wound healing. In addition, extract from Dryobalanops camphora may treat dermatitis as topical application.

One of embodiments of this current invention comprises Forsythia suspense in the herb composition. Forsythia suspense is another traditional herb medicine, and it has been demonstrated to have strong anti-bacterial, antivirus and anti-inflammatory effect. When it is applied on the top of skin, Forsythia suspense can treat dermatological disorders such as psoriasis (J M Prieto, Journal of Pharmacy and Pharmacology, 2003, 55(9), 1275-1282).

To make the said herb composition, dried root or leaves or stems of Polygonum cuspidatum is used. The plants are chopped into small pieces with 1 cm×1 cm×1 cm size. It should be understood that the plant could be chopped into any size for one skilled in the art. The pieces of chopped plants are incubated with 50-100% purified ethanol for 1-2 days. The mixture is filtered, and the extract liquid solution is used in the final products.

The part used for final product from Phellodendron amourense Ruprecht are its root or leaves or nut or stems. The fresh or dried plants are chopped into small pieces and soaked with 50-100% purified alcohol for over 1-3 days. When time is ready, the mixture is filtered, and the liquid solution is collect. On another embodiment, the similar procedure was used in the process of extract of Forsythia suspense and Dryobalanops camphora. The extract from Forsythia suspense or Dryobalanops camphora could be in the form of liquid solution for further use.

The final herb composition comprises the following component:

| Ingredient | Percentage |
|---|---|
| Polygonum cuspidatum extract | 0.1-99.9% |
| Phellodendron amourense Ruprecht extract | 0.1-99.9% |
| Forsythia suspense extract | 0.1-99.9% |
| Dryobalanops camphora extract | 0.1-99.9% |
| alpha-hydroxy acid | 0.1-99.9% |

In one embodiment, the herb composition does not have to include all of the extracts listed above. It could only include two or three or four of above herb extracts. For example, the herb composition comprises of Phellodendron amourense Ruprecht extract, Forsythia suspense extract, Dryobalanops camphora extract, alpha-hydroxy acid.

Another example herb composition comprises of Polygonum cuspidatum extract, Forsythia suspense extract, Dryobalanops camphora extract, alpha-hydroxy acid. This herb composition may also only include Phellodendron amourense Ruprecht extract, Forsythia suspense extract, Polygonum cuspidatum extract and alpha-hydroxy acid. The dose range of each individual component is from 0.1 to 99.9% of the herb composition.

When the herb composition only three components, 1-99.9% Polygonum cuspidatum extract, 1-99.9% Phellodendron amourense Ruprecht extract, and 1-99.9% alpha-hydroxy acid should be included in the herb composition. Of course, the ingredients could be any three components selected from the groups of Polygonum cuspidatum extract, Phellodendron amourense Ruprecht extract, Forsythia suspense extract, Dryobalanops camphora extract, alpha-hydroxy acid.

In general, if the herb composition inclined for cosmetic usage, such as increasing collagen synthesis and secretion, alpha-hydroxy acid should be included in this composition. When the composition inclines to treat burn, sun burn injury, physical injury or other open injury as well as dermatitis, Polygonum cuspidatum extract, Phellodendron amourense Ruprecht extract, Dryobalanops camphora extract should be included.

In another embodiment, the herb composition does not limited the five components listed as above. For one skilled in the art, other herb supplement or chemical enhancer may be added into the said composition. In one embodiment, the said herb composition can be in the form of liquid solution, cream, gel or lotion. Therefore, for one skilled in the art, other carrying vehicles could be added into the composition to make it in the form of cream, gel or lotion.

The best mode of the present invention include all of the five ingredients, which include Polygonum cuspidatum extract, Phellodendron amourense Ruprecht extract, Forsythia suspense extract, Dryobalanops camphora extract, and alpha-hydroxy acid. In one embodiment, the herb composition should be in acid, preferably the ph value should be 1-6.9. However, the said herb composition can be at any ph value. When ph value is greater than 7.0, the said herb composition may not function as well as the composition with a ph value less than 7.0. The said herb composition with higher ph value still can improve wound healing, stimulate stem cell in skin proliferation, promote collagen synthesis, and even treat dermatitis.

The herb composition is used as topical application. When this composition is used to treat burn, sun burn or other injury, the wound should be cleaned with distilled water, the herb composition is sprayed or smeared on the top of the wound. Preferably, the wound is open to environment, and heal naturally without any coverage. The herb medication is used three times every day for the burn injury at 1 or 2 degree. For the sever burn injury such as 3 degree burn, frequent changing topical medicine may avoidably cause some injury during the changing process, therefore it could change topical medicine with less times.

In the condition for cosmetic use, the products are used on the top of the skin as normal procedure for one skilled in the art. No specific care is needed.

1. A herb composition to treat burn, Sun burn, physical injury, dermatitis, or to promote skin stem cell proliferation, increase collagen synthesis, increase epidermal thickness, and increase desquamation of normal skin resulting in smoother, younger looking skin, comprises of the extract from Polygonum cuspidatum;

2. A herb composition as claim 1, further comprises of the extract from Phellodendron amourense Ruprecht;

3. A herb composition as claim 2, further comprises of extract from Forsythia suspense;

4. A herb composition as claim 1, or claim 2 or claim 3, further comprises of the extract from Dryobalanops camphora extract;

5. A herb composition as claim 1, or claim 2 or claim 3, further comprises of alpha-hydroxy acid;

6. A herb composition as claim 4, further comprises of alpha-hydroxy acid;

7. A herb composition as claim 4, the composition should be acid with ph value from 0.01-6.99;

8. A herb composition as claim 5, the composition should be acid with ph value from 0.01-6.99;

9. A herb composition as claim 6, the composition should be acid with ph value from 0.01-6.99;

10. A herb composition to treat burn, sun burn, physical injury, dermatitis, or to promote skin stem cell proliferation, increase collagen synthesis, increase epidermal thickness, and increase desquamation of normal skin resulting in smoother, younger looking skin, comprises of the extract from Phellodendron amourense Ruprecht;

11. A herb composition as claim 10, further comprises of extract from Forsythia suspense extract;

12. A herb composition as claim 10, or claim 11, further comprises of the extract from Dryobalanops camphora;

13. A herb composition as claim 10, or claim 11, further comprises of alpha-hydroxy acid;

14. A herb composition as claim 12, further comprises of alpha-hydroxy acid;

15. A herb composition as claim 13, the composition should be acid with ph value from 0.01-6.99;

16. A herb composition as claim 14, the composition should be acid with ph value from 0.01-6.99;

17. A herb composition to treat burn, sun burn, physical injury, dermatitis, or to promote skin stem cell proliferation, increase collagen synthesis, increase epidermal thickness, and increase desquamation of normal skin resulting in smoother, younger looking skin, comprises of the extract from Polygonum cuspidatum; extract from Phellodendron amourense Ruprecht; extract from Forsythia suspens; extract from Dryobalanops camphora and alpha-hydroxy acid;

18. A herb composition as claim 17, the composition should be acid with ph value from 0.01-6.99
